(19) United States
(12) Patent Application Publication
(10) Pub. No.: US 2017/0247645 A1
(43) Pub. Date: Aug. 31, 2017

(54) ENCAPSULATED FRAGRANCE MIXTURES

(71) Applicant: Symrise AG, Holzminden (DE)

(72) Inventors: Torsten Kulke, Höxter (DE); Sven Siegel, Holzminden (DE); Patrick Ott, Holzminden (DE); Anja Finke, Holzminden (DE)

(21) Appl. No.: 15/509,053

(22) PCT Filed: Sep. 4, 2015
(86) PCT No.: PCT/EP2015/070295

§ 371 Date: Mar. 6, 2017

(30) Foreign Application Priority Data
Sep. 8, 2014 (EP) 14183998.5

(51) Int. Cl. C11D 17/00; A61K 8/81; A61K 8/72; C11B 9/00; B01J 13/14; C11D 3/37; C11D 3/50; A61K 8/11; A61Q 5/12

(52) U.S. Cl. CPC C11D 17/0039; A61K 8/11; A61K 8/8158; A61K 8/72; A61Q 5/12; B01J 13/14; C11D 3/3723; C11D 3/505; C11B 9/00; A61K 2800/54

(57) ABSTRACT

The invention relates to fragrance mixtures comprising at least one 60 wt.-% fragrance molecules which are characterized such that they are oriented along a virtual axis X, Y and Z, having a dimension of X>5 angstrom, Y>3 angstrom and angstrom.

### FIELD OF THE INVENTION

The invention is in the field of fragrances and fragrance mixtures thereof.

### PRIOR ART

In the perfume industry, there is generally a constant need for novel fragrance mixtures, since consumers are to be provided constantly with new and modern fragrances with fresh fragrance notes. Fragrance mixtures having a wide variety of different fragrance notes are used in large numbers and countless variations in perfumes, scent mixtures (perfume compositions) and perfuming for a wide variety of different fields of use. Owing to the rising consumer demand for constantly new fragrance mixtures with new fragrance notes, there is a constant need in the perfume industry for novel fragrance mixtures that achieve novel effects, such that new fashion trends can be created in this way.

In spite of a multitude of already existing fragrance mixtures, there is a constant need in the perfume industry, for creation of novel modern perfume compositions, for novel fragrance mixtures having particular olfactory properties suitable for serving as a basis for the composition of novel modern perfumes having complex character. More particularly, the focus in the case of novel fragrance mixtures is directed primarily to their having, over and above their primary properties, namely their olfactory properties, additional positive secondary properties, for example higher stability under particular use conditions, high abundance, high radiance, good diffusivity (i.e. good spatial effect), fullness, power and/or naturalness, odor-boosting properties or else better dermatological compatibility, good solubility, and toxicological compatibility and biodegradability.

Combinations of fragrances which together give a fragrance mixture that are pleasing to the consumer and that have the abovementioned secondary properties are always difficult to identify, since the mechanisms of olfactory perception are firstly not sufficiently well known, nor have the connections between specific olfactory perception on the one hand and the chemical structure of the corresponding fragrance on the other hand been sufficiently well researched, and so it is frequently the case that even slight changes in the structure of a known fragrance cause significant changes in the sensory properties and impairments of compatibility for the human organism.

DE 10 2012 221619 A1 (HENKEL) discloses fragrance mixtures comprising hexyl salicylate and additionally at least one further salicylate. The salicylates are used in an amount of 5% to 100% by weight, especially 10% to 30% by weight—based on the mixture.

The example discloses two fragrance mixtures respectively comprising, as well as hexyl salicylate, i-amyl salicylate and cyclohexyl salicylate.

EP 1767185 A1 (TAKASAGO) discloses core-shell capsules comprising 50%-100% of a fragrance composition which is produced by mixing at least two perfume additives.

EP 2204155 A1 (TAKASAGO) discusses a fragrance composition in the form of core-shell capsules which contains 20%-100% by weight of at least one cyclic fragrance material, for example ethyl 3-methyl-3-phenylglycidate or citronellylnitrile.

EP 2620211 A1 (TAKASAGO) relates to a microcapsule having a core consisting of emulsifiable fragrances, wherein the fragrances are a combination of four different fragrances.

WO 2010 142815 A2 (SYMRISE) provides a fragrance mixture comprising 34.98%-99% by weight of 2,2-dimethyl-3-(3 methylphenyl)propanol and 1%-30% by weight of 4,4a,5,9b-tetrahydro-2,4-dimethylindenol [1,2d]-m-dioxin, and optionally 4 further compounds, namely 2-isobutyl-4-methyltetrahydro 2/-/-pyran 4 ol, citronellyloxyacetaldehyde, 4-n-decylpyridines and 4-(4-methyl-3-penten-1-yl)-3-cyclohexenecarbaldehyde.

WO 2013 109798 A2 (P&G) provides a consumer product containing 0.0001% to 25% by weight of one or more perfume raw materials—based on the consumer product. Table 1 discloses 22 usable fragrance starting materials.

US 2010/137178 A1 (SMETS) discloses a multitude of perfume raw materials that are listed in table 1. In order to produce different consumer products, one or more fragrance starting materials from table 1 are used.

US 2014/161740 A1 (DENUTTE) discloses perfume raw materials having the following structure:

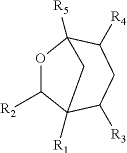

in which R₁, R₄ and R₅ are each independently selected from the group consisting of: a linear or branched C₁-C₆-alkyl group; a linear or branched C₂-C₆-alkenyl group; and a linear or branched C₂-C₆-alkyne unit; and where R2 and R3 are each independently selected from the group consisting of: hydrogen, a linear or branched C₁-C₆-alkyl group; a linear or branched C₂-C₆-alkenyl group; and a linear or branched C2-C₆-alkyne unit.

There is therefore fundamentally a constant need in the perfume industry for new fragrance mixtures suitable for production of olfactory compositions or perfumed articles. More particularly, there is a need for fragrance mixtures which, by virtue of the abovementioned technical properties, lead to an increased benefit of fragrance compositions and perfume oils.

It was therefore an object of the present invention to detect individual fragrances that are particularly suitable for combination to fragrance mixtures. It was a particular object of the present invention to find a general definition for fragrances which can be mixed together to give stable fragrance mixtures and can subsequently be encapsulated without any great problems. More particularly, the new fragrance mixtures are to be configured such that they can be encapsulated by a wide variety of different encapsulation processes known in the prior art, with a wide variety of different carrier materials, without occurrence of any great or, if any, only minor losses of the fragrances as a result of diffusion or the like in the encapsulation in the aqueous medium or in the drying. Moreover, the fragrance mixtures that have thus been encapsulated are to have a long-lasting fragrance and high-impact, meaning that rapid olfactory perception follows the breakup or dissolution of the capsules.

### DESCRIPTION OF THE INVENTION

The invention therefore provides fragrance mixtures comprising at least 60% by weight, preferably at least 70% by weight and more preferably at least 80% by weight of fragrances, characterized in that the fragrance molecules, if they were aligned along a virtual set of axes X, Y and Z, have dimensions of X>5 angströms, Y>3 angströms and Z>2 angströms, the fragrance molecules preferably having dimensions of X>7 angströms, Y>4 angströms and Z>2 angströms, with the proviso that the number of different fragrances in the mixture is at least 2.

In the context of the invention, the fragrances may also simultaneously be aromas.

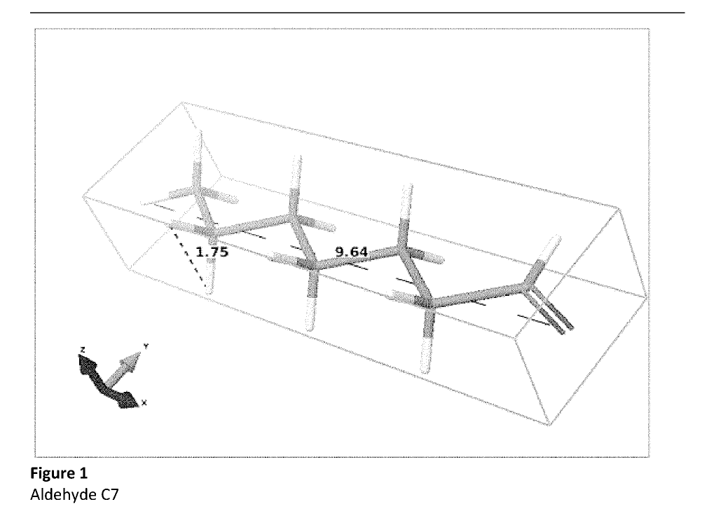

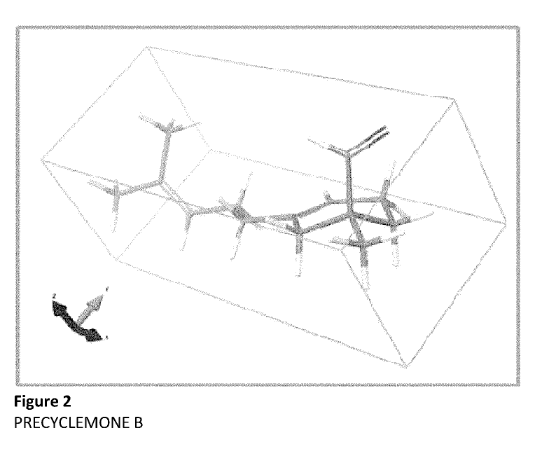

In the detection of particularly suitable fragrances for fragrance mixtures and subsequent encapsulation thereof, the structures of individual fragrance molecules were subjected to force field energy minimization taking account of their stereochemistry, and aligned along a virtual set of axes X, Y and Z for further calculation. Subsequently, a virtual box was created around the individual structures and the dimensions of the box were recorded for further calculation (see FIGS. 1 and 2). It was found that, surprisingly, the fragrances thus detected are of particularly good suitability for combination and mixed together to give fragrance mixtures, and are particularly advantageous since they can subsequently be encapsulated without any problem.

Preferably, a fragrance mixture of the invention therefore comprises at least 80% by weight of fragrances which, if they were aligned along a virtual set of axes X, Y and Z, have dimensions of X>7 angströms, Y>4 angströms and Z>2 angströms.

In a preferred embodiment, it has been found that it is particularly advantageous when the individual fragrances in the fragrance mixture of the invention have an odor threshold value (OTV) of less than 10 ppm (in air), preferably of less than 5 ppm, more preferably of less than 2 ppm.

The odor threshold is the threshold at which a fragrance or odorant is olfactorily perceived by an organism. The odor threshold value, OTV for short, is that minimum concentration of a particular gaseous, sensorily active substance in the surrounding medium that this lifeform is just able to perceive through its sense of smell. In the present invention, preference is given to fragrances having a (very) low odor threshold value. The lower this is, the lower the concentration at which the fragrance is then perceived.

In a further preferred embodiment of the present invention, the number of different fragrances in a fragrance mixture of the invention is at least two and not more than 10. Preferably, a fragrance mixture of the invention has three, four, five, six, seven, eight or nine different fragrances. Preference is given to fragrance mixtures having 5, 6, 7 or 8 different or fragrances, very particular preference to mixtures having 6, 7 or 8 different fragrances.

In addition, in a preferred execution, in the fragrance mixture of the invention, at least one of the fragrances is present in the composition in a concentration of at least 10% by weight, preferably at least 15% by weight, more preferably at least 20% by weight, and preferably not more than 60% by weight, more preferably not more than 50% by weight. Preferably, the concentration of at least two of the fragrances is at least 15% by weight.

In a fragrance mixture of the invention, preferably at least one of the fragrances has a sulfur or nitrogen atom, preferably in the form of a nitrile group.

In addition, it is advantageous in a fragrance mixture of the invention when at least one of the fragrances has a molar mass of greater than 120 g/mol.

In a further preferred embodiment, the individual fragrances of the fragrance mixture of the invention have a log K_{ow} value of 1 to 10, preferably of 1.5 to 8, more preferably of 2 to 5.5.

The log K_{ow} (octanol/water partition coefficient) is a measure of the lipophilicity of a substance. In terms of calculation, it is the partition coefficient K_{ow} of a substance in a mixture of 1-octanol and water. K_{ow} is greater than one when a substance has better solubility in fat-like solvents such as n-octanol, and less than one when it has better solubility in water. Correspondingly, log K_{ow} is positive for lipophilic substances and negative for hydrophilic substances.

The special feature of the present invention is the combination of the different abovementioned parameters for a fragrance molecule. Fragrances having the abovementioned parameters can be combined particularly efficiently with one another to give a fragrance mixture which show advantages such as good stability, good encapsulation properties and the necessity for further solvents minimized.

It has been found that the fragrance mixtures of the present inventions can be formulated and encapsulated even without solvents.

Accordingly, in a preferred embodiment, the fragrance mixture of the invention contains only very little solvent, if any.

“Very little solvent” is accordingly understood to mean a content of less than 30% by weight and preferably less than 20% by weight of solvent in a fragrance mixture of the invention.

As already mentioned, it has also been found that, surprisingly, the inventive fragrance mixtures of the present invention are particularly advantageous, since they can be encapsulated without any problem in a wide variety of different encapsulation processes using a wide variety of different carrier materials. More particularly, it has been found that the fragrance mixtures of the invention, in the encapsulation in aqueous media, have only minor losses as a result of diffusion, if any. It was likewise found that the fragrances are barely or insignificantly lost in the drying of the capsules.

Therefore, the present invention further provides capsules comprising the inventive fragrance mixtures of the abovementioned type.

The capsules preferably have an average diameter of 1 μm to 8 mm, preferably 5 μm to 2 mm, more preferably 10 μm to 500 μm. The capsule sizes are not restricted and can be produced in a variable manner if required according to the field of application and use.

Accordingly, in a preferred embodiment, the capsules, as required, may assume any average diameter between 1 μm and 8 mm.

Encapsulation

Encapsulation techniques for fragrances are known from the prior art. Typically, encapsulations are effected by, for example, methods such as extrusion, embedding in gelatin capsules, for example, spray-drying, agglomeration (wet granulation in mixers or fluidized bed agglomeration), or by fluidized bed spray granulation. A further possible method of production for encapsulation is that of molecular weight-increasing granulation in the fluidized bed, as published in DE 199 56 604 A1. Here, a process is presented in which the liquid formulation is atomized into a fluidized bed by spray nozzles. The process employs a fluidized bed process according to EP-A-0163836 and EP-A-0332929.

Methods of this kind are well known and produce fragrance capsules having defined product qualities. These fragrance capsules are utilized to an ever greater degree in a multitude of branches of industry. This relates both to the volumes produced and to the wide variety of different formulations.

Typically, encapsulations are effected with the aid of solid coating materials, for example starches, including the degradation products thereof and chemically or physically produced derivatives (especially dextrins and maltodextrins), gelatin, gum arabic, agar-agar, ghatti gum, gellan gum, modified and unmodified celluloses, pullulan, curdlan, carrageenans, alginic acid, alginates, pectins, inulin, xanthan gum and mixtures of two or more of these substances.

The solid encapsulation material is preferably a gelatin (especially porcine gelatin, bovine gelatin, poultry gelatin and/or fish gelatin), the latter preferably having a threshold factor of not less than 20, preferably not less than 24. Likewise preferred are maltodextrins (especially based on cereals, specifically maize, wheat, tapioca or potatoes), preferably having DE values in the range from 10 to 20. Preference is further given to celluloses (e.g.

cellulose ethers), alginates (e.g. sodium alginate), carrageenan (e.g. beta-, iota-, lambda- and/or kappa-carrageenan), gum arabic, curdlan and/or agar-agar. Corresponding capsules are, for example, in the following publications in detail EP 0389700 A1, U.S. Pat. No. 4,251,195, U.S. Pat. No. 6,214,376, WO 2003 055587 or WO 2004 050069 A1.

The capsules may alternatively also be in the form of microcapsules. The terms “microcapsule” or “nanocapsule” are understood by those skilled in the art to mean spherical aggregates having a diameter in the range from about 0.0001 to about 5 and preferably 0.005 to 0.5 mm, containing at least one solid or liquid core surrounded by at least one continuous shell. More specifically, they are finely dispersed liquid or solid phases which have been ensheathed by film-forming polymers and wherein the preparation involves precipitation of the polymers, after emulsification and coacervation or interfacial polymerization, on the material to be ensheathed. In another method, molten waxes are incorporated in a matrix (“microsponge”) which, in the form of microparticles, may additionally be ensheathed by film-forming polymers. In a third method, particles are coated alternately with polyelectrolytes of different charge (“layer-by-layer” method). The microscopically small capsules can be dried like powders. As well as single-core microcapsules, also known are multicore aggregates, also called microspheres, containing two or more cores distributed in a continuous shell material. Single- or multicore microcapsules may additionally be ensheathed by an additional second, third etc. shell. The shell may consist of natural, semisynthetic or synthetic materials. Natural shell materials are, for example, gum arabic, agar-agar, agarose, maltodextrins, alginic acid or salts thereof, e.g. sodium alginate or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes. Semisynthetic shell materials include chemically modified celluloses, especially cellulose esters and ethers, e.g. cellulose acetate, ethyl cellulose, hydroxypropyl cellulose, hydroxypropyl methyl cellulose and carboxymethyl cellulose, and also starch derivatives, especially starch ethers and esters. Synthetic shell materials are, for example, polymers such as amino resins, phenolic resins, polyureas, polyacrylates, polyamides, polyvinyl alcohols or polyvinyl pyrrolidone.

Examples of prior art microcapsules are the following commercial products (the shell material is specified in brackets in each case): Hal/crest Microcapsules (gelatin, gum arabic), Coletica Thalaspheres (maritime collagen), Lipotec Millicapsules (alginic acid, agar-agar), Induchem Unispheres (lactose, microcrystalline cellulose, hydroxypropyl methyl cellulose); Unicerin C30 (lactose, microcrystalline cellulose, hydroxypropyl methyl cellulose), Kobo Glycospheres (modified starch, fatty acid esters, phospholipids), Softspheres (modified agaragar) and Kuhs Probiol Nanospheres (phospholipids), and also Primaspheres and Primasponges (chitosan, alginates) and Primasys (phospholipids).

A particularly preferred form of encapsulation is to introduce the fragrances into a matrix and then to envelop them with a shell. For this purpose, the prior art includes a whole series of methods.

One example is what are called chitosan microcapsules, the preparation process for which is sufficiently well known from the prior art [WO 01/01926, WO 01/01927, WO 01/01928, WO 01/01929]. Microcapsules having mean diameters in the range from 0.0001 to 5, preferably 0.001 to 0.5 and especially 0.005 to 0.1 mm, consisting of a shell membrane and a matrix containing the active ingredients, can be obtained, for example, by

using gel formers, cationic polymers and active ingredients to form a matrix,

optionally dispersing the matrix in an oil phase,

treating the dispersed matrix with aqueous solutions of anionic polymers and optionally removing the oil phase at the same time.

Steps (a) and (c) are interchangeable in that anionic polymers are used in place of the cationic polymers in step (a) and vice versa.

It is also possible to produce capsules by ensheathing the active ingredient alternately with layers of differently charged polyelectrolytes (layer-by-layer technology). In this connection, reference is made to European Patent EP 1064088 B1 (Max-Planck Gesellschaft).

It is often the case with fragrances encapsulated in this way that they are only released in a retarded manner or by means of an external trigger (e.g. water, shear forces, change in pH), such that the fragrance perception (“impact”) in the first seconds or before the action of the trigger is low.

It has been found that the encapsulated fragrance mixtures of the invention exhibited a high impact, meaning that the fragrance could be strongly perceived, such that a first strong odor impression is produced.

It has additionally been found that, surprisingly, capsules containing the fragrance mixtures of the invention could be incorporated in clear applications, and that this did not lead to any cloudiness of the formulation. Advantageously, the fragrance mixtures of the invention do not diffuse out of the capsule in aqueous media, especially those containing surfactants or emulsifiers, and so these are stable to some degree.

It has also been found that the fragrance mixtures thus encapsulated remain stable in harsh environments in the presence of, for example, oxidizing agents or bleaches, and in high- or low-pH environments. As a result, the fragrance mixtures of the invention are particularly suitable for incorporation into products where such conditions exist, for example into washing and cleaning compositions or cosmetic products.

Accordingly, a further aspect of the present invention is consumer products comprising the fragrance mixtures of the invention or capsules comprising the fragrance mixture of the invention.

### INDUSTRIAL APPLICABILITY

A further aspect of the present invention is the use of the fragrance mixture of the invention in consumer products.

Accordingly, the present invention further provides consumer products comprising a fragrance mixture of the invention and/or capsules comprising the fragrance mixture of the invention. In this context, consumer products within the scope of the present invention are different products, for example those from the fields of washing and cleaning compositions, cosmetic products, perfume articles and cosmetic cleaning compositions that are preferably selected from the group consisting of:

perfume extracts, eau de parfums, eau de toilettes, aftershaves, eau de colognes, pre-shave products, splash colognes, perfumed freshen-up wipes, perfumes for acidic, alkaline and neutral cleaning compositions, washing compositions, washing tablets, disinfectants, and of air fresheners, aerosol sprays, waxes and polishes, and also personal care products, bath oils, cosmetic emulsions, for example skin creams and skin lotions, sunscreen creams and lotions, aftersun creams and lotions, hand creams and lotions, foot creams and lotions, depilatory creams and lotions, aftershave creams and lotions, tanning creams and lotions, haircare products, for example hairsprays, hair gels, hairsetting lotions, hair rinses, hair colorants, hair-shaping products and hair-straightening products, hair tonics, hair creams and hair lotions, deodorants and antiperspirants, decorative cosmetics products, for example eyeshadows, nail varnishes, make-up products, lipsticks and mascara, and also candles, lamp oils, joss sticks, insecticides, repellents and propellants.

Fragrance mixtures of the invention and capsules that comprise them may generally be used (for example in concentrated form, in solutions or in modified form as described below) for the production of, for example, perfume extracts, eau de parfums, eau de toilettes, aftershaves, eau de colognes, pre-shave products, splash colognes and perfumed freshen-up wipes, and the perfuming of acidic, alkaline and neutral cleaning compositions, such as floor cleaners, window glass cleaners, dishwashing products, bath and sanitary cleaners, scouring cream, solid and liquid toilet cleaners, carpet cleaners in powder and foam form, liquid washing compositions, pulverulent washing compositions, laundry pre-treatment compositions such as bleaches, soaking compositions and stain removers, fabric softeners, washing soaps, washing tablets, disinfectants, surface disinfectants, and of air fresheners in liquid form, of the gel type or in a form applied to a solid carrier, aerosol sprays, waxes and polishes, such as furniture polishes, floor waxes, shoe creams, and personal care products, for example solid and liquid soaps, shower gels, shampoos, shaving soaps, shaving foams, bath oils, cosmetic emulsions of the oil-in-water, of the water-in-oil and of the water-in-oil-in-water type, for example skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, haircare products, for example hairsprays, hair gels, setting hair lotions, hair rinses, permanent and semipermanent hair colorants, hair-shaping products such as cold waves and hair-straightening products, hair tonics, hair creams and hair lotions, deodorants and antiperspirants, for example underarm sprays, roll-ons, deodorant sticks, deodorant creams, decorative cosmetics products, for example eyeshadows, nail varnishes, make-up products, lipsticks and mascara, and also candles, lamp oils, joss sticks, insecticides, repellents and propellants.

### EXAMPLES

### Examples 1 TO 10, Comparative Examples C1 TO C3

### Calculations of the Fragrance Molecules

The structures of individual fragrance molecules were subjected to force field energy minimization, taking account of the stereochemistry, provided that this is known with certainty, and aligned along a virtual set of axes for further calculation. Subsequently, a virtual box was created around the particular structures and the size of the box was recorded for further calculation (unit of measurement: angströms). Examples 1 to 10 are in accordance with the invention; examples C1 to C3 serve for comparison.

Software: from Schrödinger LLC
Maestro Version 9.5.014
Module: MacroModel
Force field: OPLS2005
Parameters: gas phase, constant dielectric

TABLE 1
Data for the fragrances calculated that are usable in accordance with the invention

| | | | Dimensions | | | | | |
|---|---|---|---|---|---|---|---|---|
| | | | [angstroms] | | | OTV | log | Weight |
| Ex. | Compound | Structure | x | y | z | ppm | Kow | mol/g |
| 1 | Rose oxide (FEMA 3236) | 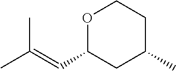 | 9.11 | 4.76 | 4.55 | 0.5 | 3.58 | 154.3 |
| 2 | Aldehyde C16 (FEMA 2444) | 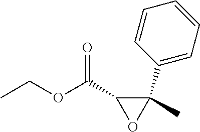 | 8.07 | 7.16 | 4.82 | 0.2 | 3 | 206.2 |
| 3 | Agrunitrile (citronellylnitrile, CAS 51566-62-2) | 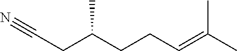 | 10.56 | 4.01 | 3.43 | 1 | 3.55 | 151.3 |
| 4 | Methyl octyne carbonate (CAS 111-80-8) | 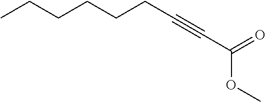 | 12.04 | 6.29 | 2.24 | 0.2 | 3.1 | 168.2 |
| 5 | Bucco leaf oil | 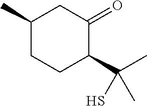 | 7.67 | 5.64 | 4.64 | 0.2 | | |
| 6 | Pre-cyclemone B (CAS 52474-60-9) | 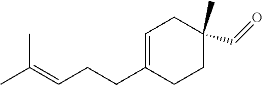 | 11.58 | 4.87 | 4.40 | 0.5 | 5.19 | 206.3 |
| 7 | Allyl amyl glycolate (CAS 67634-00-8) | 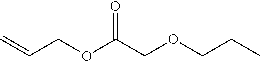 | 11.72 | 5.24 | 4.56 | 0.1 | 2.34 | 186.3 |
| 8 | Thiocineol (FEMA 4108) | 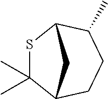 | 7.02 | 5.29 | 4.93 | <0.01 | 3.95 | 170.3 |
| 9 | 2-Isobutyl-3- methoxypyrazine (FEMA 3132) | 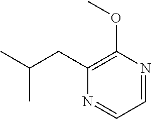 | 7.50 | 6.99 | 4.47 | 0.1 | 2.86 | 166.2 |
| 10 | cis-4-Decenal (FEMA 3264) | 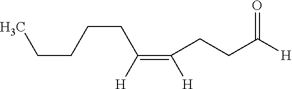 | 11.50 | 5.83 | 3.50 | 0.2 | 3.55 | 154.3 |
| C1 | Aldehyde C-7 (FEMA 2540) | 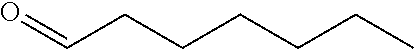 | 9.64 | 2.59 | 1.73 | | | 114.2 |
| C2 | Ethyl butyrate (FEMA 2427) | 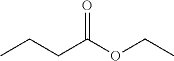 | 9.20 | 2.93 | 1.76 | | | 116.2 |
| C3 | Diphenyl oxide (CAS 32576-61-7) | 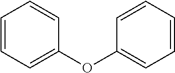 [SMILES: c1ccc(Oc2ccccc2)cc1] | 10.08 | 4.81 | 0.0 | | | 170.2 |

### Use Examples 1: Fragrance Mixtures I TO V

The suitable fragrances from table 1 were mixed in different formulations to give fragrance mixtures I to V and then encapsulated.

Production of the Capsules

A cylindrical 1 L stirred vessel with an installed stirrer unit (dissolver) was initially charged, while stirring gently, with 200 g of water, 35 g of a 20% by weight aqueous solution of a protective colloid, 40.5 g of a 70% by weight aqueous solution of the melamine-formaldehyde resin mixture (e.g. Luracoll SD from BASF).

Then the stirrer speed of the dissolver was increased to such an extent that good mixing was achieved. Still while mixing, 200 g of fragrance oil mixture to be encapsulated were then added gradually. The mixture was adjusted to 30-40° C. and the dissolver speed was set to 1200 rpm. Subsequently, X g of 10% by weight formic acid were added and the dissolver speed was reduced to 1000 rpm. After about 20 min, capsules formed. As soon as the desired capsule size had been attained, the speed of rotation of the dissolver was lowered to 1000 rpm and the capsule dispersion was stirred at 30-40° C. and 1000 rpm for half an hour.
The results are compiled in table 2.

TABLE 2
Inventive fragrance mixtures I to V

| | Fragrance mixtures | | | | |
|---|---|---|---|---|---|
| Compound | I | II | III | IV | V |
| Rose oxide | 50 | 50 | 50 | 50 | 100 |
| Aldehyde C16 | — | — | — | 450 | — |
| Agrunitrile | 200 | 195 | 195 | 20 | 200 |
| Diphenyl oxide | — | — | — | — | 2 |
| Bucco leaf oil | — | — | — | 2 | — |
| Precyclemone B | 300 | 280 | 290.5 | — | 260 |
| Allyl amyl glycolate | 400 | 380 | 392 | 450 | 400 |
| Thiocineol | — | 40 | 20 | 60 | |
| 2-Isobutyl-3- | — | 5 | 2.5 | — | 8 |
| methoxypyrazine | | | | | |
| cis-4-Decenal | 50 | 50 | 50 | 18 | 30 |
| Total amount | 1000 | 1000 | 1000 | 1000 | 1000 |

### Use Example 2

Stability Test of the Encapsulated Fragrance Mixtures I to V

The resultant capsule dispersions were extracted with acetone to determine the free (unencapsulated) oil content, and the concentration of the fragrance mixtures in the extract obtained was determined.

To determine the total oil content, the resultant capsule dispersions were exhaustively extracted with a suitable solvent (e.g. ethanol, acetone). This extract was made up to a known volume and the content of the fragrance mixture was determined.

The results are compiled in table 3.

TABLE 3
Determination of the capsule quality (all figures in % by weight)

| | | | Total content of |
|---|---|---|---|
| | | | average amount of |
| Capsules with | Free oil, | Oil, | fragrance in a |
| fragrance mixture | unencapsulated* | total amount | capsule |
| I | 0.00 | 0.0 | 33.0 |
| II | 0.02 | 0.86 | 27.9 |
| III | 0.00 | 0.14 | 31.7 |
| IV | 0.00 | 0.16 | 34.5 |
| V | 0.00 | 0.00 | 33.7 |

### Use Example 3

### Stability Test in Fabric Softeners

A commercial perfume-free fabric softener formulation having an ester quat content of about 15% was admixed with 0.5 g of the encapsulated fragrance mixtures I to V and stored at 40° C. After a storage time of 1, 4, 8 and 12 weeks, the content of the odorants that had diffused into the bulk was determined with the aid of headspace measurements in the air phase above. The results are compiled in table 4. What is reported is the percentage of the oil still remaining in the capsule.

TABLE 4
Stability test (figures in % in relation to the starting formulation)

| | |
|---|---|
| Capsules with | Stability of the capsules |
| fragrance | in the fabric softener at 40° C. |

| mixture | 1 week | 4 weeks | 8 weeks | 12 weeks |
|---|---|---|---|---|
| I | 100 | 97 | 74 | 35 |
| II | 89 | 82 | 58 | 24 |
| III | 99 | 91 | 55 | 25 |
| IV | 100 | 97 | 69 | 47 |
| V | 100 | 94 | 57 | 11 |

### Use Example 4

### Impact of the Capsules Comprising Fragrance Mixtures I to V

A commercial perfume-free fabric softener formulation of the Vernell® type was admixed with 0.01% by weight of the encapsulated fragrance mixtures Ito V. Subsequently, about 20 g of this mixture were washed onto 2 kg of terrycloth towels in a standard European domestic washing machine. After the spin cycle, the towels were removed and dried. Subsequently, the towels were rubbed repeatedly by a panel consisting of 4 experienced testers and the intensity of the olfactory evolution (“impact”) was assessed on a scale from 1 (weak) to 10 (very strong). The results are compiled in table 5. What are reported are the mean values from 3 successive test series.

TABLE 5
Impact

| | |
|---|---|
| Capsules with | |
| fragrance | Odor intensity |

| mixture | Tester 1 | Tester 2 | Tester 3 | Tester 4 |
|---|---|---|---|---|
| I | 8 | 7 | 7 | 8 |
| II | 7 | 6 | 7 | 7 |
| III | 8 | 8 | 7 | 5 |
| IV | 8 | 9 | 8 | 8 |
| V | 8 | 9 | 7 | 7 |

### Formulation Examples

TABLE 6
Bar soaps

| Composition | A1 | A2 | A3 |
|---|---|---|---|
| Sodium C12/14 Olefin Sulfonate | 40.0 | — | — |
| Disodium Lauryl Sulfosuccinate | — | 40.0 | — |
| Lauryl Glucoside | 15.0 | 15.0 | 55.0 |
| Cetylstearyl Alcohol | 12.0 | 12.0 | 12.0 |
| Paraffin Oil (m.p. 54 to 56° C.) | 8.0 | 8.0 | 8.0 |
| Maize Starch, degraded | 8.0 | 8.0 | 8.0 |
| Palmitic Acid (and) Stearic Acid | 5.5 | 5.5 | 5.5 |
| Glyceryl Stearate | 2.0 | 2.0 | 2.0 |
| Coco Glyceride | 2.0 | 2.0 | 2.0 |
| Polyquaternium-7 | 1.0 | 1.0 | 1.0 |
| Perfume oil | 1.0 | 1.0 | 1.0 |
| Titanium dioxide | 0.5 | 0.5 | 0.5 |
| Capsules with fragrance mixture I | 0.5 | 0.5 | 0.5 |

| | |
|---|---|
| Water | ad 100 |

TABLE 7
Syndet soaps

| Composition | B1 | B2 | B3 |
|---|---|---|---|
| Lauryl Glucoside | 15.0 | 15.0 | 15.0 |
| Sodium C12/14 Olefin Sulfonate | 40.0 | 40.0 | — |
| Sodium Lauryl Sulfate | — | — | 40.0 |
| Paraffin Oil (m.p. 54° C.) | 8.0 | 8.0 | 8.0 |
| Tallow Fatty Alcohol | 7.0 | 7.0 | 7.0 |
| Maize starch | 17.0 | — | 17.0 |
| Dextrose | — | 17.0 | — |
| Coco Fatty Acids | 10.0 | 10.0 | 10.0 |
| Titanium dioxide | 1.0 | 1.0 | 1.0 |
| Capsules with fragrance mixture II | 0.5 | — | 0.5 |
| Capsules with fragrance mixture III | — | 0.5 | — |

| | |
|---|---|
| Water | ad 100 |

TABLE 8
Universal cleaner in gel form

| Composition | C1 | C2 | C3 | C4 | C5 | C6 |
|---|---|---|---|---|---|---|
| Lauryl Glucoside | 5.0 | 1.5 | 1.5 | 1.5 | 5.0 | 1.5 |
| Decyl Glucoside | — | 3.5 | 3.5 | 3.5 | — | 3.5 |
| Sodium Lauryl Sulfate | 9.0 | 6.0 | 3.0 | — | 9.0 | 6.0 |
| Sodium Laureth Sulfate | — | 3.0 | — | — | — | 3.0 |
| Cocamidopropyl Betaine | — | — | 6.0 | 9.0 | — | — |
| Ethanol | 3.5 | 3.5 | 3.5 | 3.5 | 3.5 | 3.5 |
| Citric Acid Monohydrate | 3.6 | 3.6 | 3.6 | 3.6 | 3.6 | 3.6 |
| Sodium Hydroxide | 0.5 | 0.5 | 0.5 | 0.5 | 0.5 | 0.5 |
| Laurylamine + 1EO | 4.5 | 3.0 | 5.0 | 2.5 | — | — |
| Hexanediol-1,6 | — | 0.5 | — | | 0.5 | — |
| Capsules with fragrance mixture IV | 0.5 | — | 0.5 | — | 0.5 | — |
| Capsules with fragrance mixture V | — | 0.5 | — | 0.5 | — | 0.5 |

| | |
|---|---|
| Water | ad 100 |

TABLE 9
Powder washing composition

| Composition | E1 | E2 | E3 | E4 | E5 | E6 |
|---|---|---|---|---|---|---|
| Sodium Laureth-1 Sulfate | 12.0 | 8.0 | 8.0 | 4.0 | 4.0 | — |
| Dodecylbenzene Sulfonate | — | — | 4.0 | 8.0 | 4.0 | 8.0 |
| Sodium Lauryl Sulfate | — | 4.0 | — | — | 4.0 | 4.0 |
| Coco Fatty Acid, Sodium Salt | 1.0 | 1.0 | 1.0 | 1.0 | 1.0 | 1.0 |
| Coco Glucosides | — | — | — | 7.0 | — | — |
| Laureth-5 | 7.0 | 7.0 | 7.0 | — | 7.0 | 7.0 |
| Zeolite A | 24.0 | 24.0 | 24.0 | 24.0 | 24.0 | 24.0 |
| Sodium silicate, amorphous | 3.0 | 3.0 | 3.0 | 3.0 | 3.0 | 3.0 |
| (modulus 1:2) | | | | | | |
| Sodium carbonate | 12.0 | 12.0 | 12.0 | 12.0 | 12.0 | 12.0 |
| Polymeric polycarboxylate | 5.5 | 5.5 | 5.5 | 5.5 | 5.5 | 5.5 |
| Perborate monohydrate | 16.0 | 16.0 | 16.0 | 16.0 | 16.0 | 16.0 |
| Tetracetylethylenediamine | 5.5 | 5.5 | 5.5 | 5.5 | 5.5 | 5.5 |
| Carboxymethylcellulose | 0.3 | 0.3 | 0.3 | 0.3 | 0.3 | 0.3 |
| Optical brightener | 0.2 | 0.2 | 0.2 | 0.2 | 0.2 | 0.2 |
| Enzyme granulate (protease) | 1.0 | 1.0 | 1.0 | 1.0 | 1.0 | 1.0 |
| Capsules with fragrance | 0.5 | — | 0.5 | — | 0.5 | — |
| mixture I | | | | | | |
| Capsules with fragrance | — | 0.5 | — | 0.5 | — | 0.5 |
| mixture V | | | | | | |

| | |
|---|---|
| Water | ad 100 |

TABLE 10
Manual dishwashing composition

| Composition | F1 | F2 | F3 | F4 |
|---|---|---|---|---|
| Sodium Laureth-1 Sulfate | 15 | 15 | 15 | 15 |
| Sodium Laureth-2 Sulfate | 15 | — | — | — |
| Sodium Lauryl Sulfate | — | 15 | — | — |
| Coco Glucosides | — | — | 15 | — |
| Sodium Octyl Sulfosuccinate | — | — | | 15 |
| Cocamidopropyl Betaine | 1 | 3 | 3 | 3 |
| Capsules with fragrance mixture III | 0.5 | — | 0.5 | — |
| Capsules with fragrance mixture IV | — | 0.5 | — | 0.5 |

| | |
|---|---|
| Water | ad 100 |

TABLE 11
Cleaning composition

| Compositon | G1 | G2 | G3 | G4 | G5 | G6 |
|---|---|---|---|---|---|---|
| C_{12/16}-Alkyl sulfate sodium salt | 5.0 | 5.0 | 4.0 | 0 | 5.0 | 0 |
| C_{16/18}-Tallow fatty alcohol + 25EO | 3.0 | 3.0 | 4.0 | 0 | 0 | 5.0 |
| C_{12/14}-Coconut alkyl oligoglucoside | 1.0 | 2.0 | 2.0 | 5.0 | 0 | 0 |
| Sodium metasilicate | 15.0 | 15.0 | 15.0 | 15.0 | 15.0 | 15.0 |
| Sodium carbonate | 26.0 | 24.0 | 24.0 | 30.0 | 30.0 | 30.0 |
| Sulfonated styrene/MA copolymer, | — | 2.0 | 2.0 | — | — | — |
| sodium salt | | | | | | |
| Capsules with fragrance mixture II | 0.5 | — | 0.5 | — | 0.5 | — |
| Capsules with fragrance mixture IV | — | 0.5 | — | 0.5 | — | 0.5 |

| | |
|---|---|
| Sodium tripolyphosphate | ad 100 |

TABLE 12
Cosmetic creams

| Composition | H1 | H2 | H3 | H4 | H5 |
|---|---|---|---|---|---|
| Glyceryl Sterate (and) Ceteareth 12/20 | 5.0 | 5.0 | 4.0 | — | — |
| (and) Cetearyl Alcohol (and) Cetyl | | | | | |
| Palmitate | | | | | |
| Ceteareth-12 | — | — | 1.0 | — | — |
| Polyglyceryl-3 Isostearate | — | — | — | 4.0 | — |
| Polyglyceryl-2 Dipolyhydroxystearate | — | — | — | — | 4.0 |
| Glyceryl Oleate | — | — | — | 2.0 | — |
| PEG-7 Glyceryl Cocoate | — | — | — | — | 2.0 |
| Dicaprylyl Ether | — | — | — | 5.0 | 6.0 |
| Hexyldecanol (and) Hexyldecyl Laurate | — | — | 3.0 | 10.0 | 9.0 |
| Cetearyl Isononanoate | 3.0 | 3.0 | — | — | — |
| Decyl Oleate | 3.0 | 3.0 | — | — | — |
| Coco Caprylate Caprate | — | — | 3.0 | 5.0 | 5.0 |
| Beeswax | — | — | — | 7.0 | 5.0 |
| Hydrolyzed Elastin | 2.0 | — | — | — | — |
| Hydrolyzed Collagen | — | 2.0 | — | — | — |
| Hydrolyzed Wheat Gluten | — | — | 0.5 | — | — |
| Sodium Cocoyl Hydrolyzed Wheat Protein | — | — | — | 0.5 | 0.5 |
| Glycol Distearate (and) Laureth-4 (and) | 5.0 | 5.0 | 4.0 | — | — |
| Cocamidopropyl Betaine | | | | | |
| Glycerol (86% strength by weight) | — | — | 1.0 | — | — |
| Capsules with fragrance mixture III | 0.5 | — | 0.5 | — | 0.5 |
| Capsules with fragrance mixture V | — | 0.5 | — | 0.5 | — |

| | |
|---|---|
| Water | ad 100 |

1. A fragrance mixture comprising at least 60% by weight of fragrance molecules, wherein, when they are aligned along a virtual set of axes X, Y and Z, they have dimensions of X>5 angströms, Y>3 angströms and Z>2 angströms, with the proviso that the number of different fragrances in the mixture is at least 2.

2. The fragrance mixture as claimed in claim 1, wherein the odor threshold value (OTV) of the individual fragrance molecules in the mixture is less than 10 ppm.

3. The fragrance mixture as claimed in claim 1, wherein the number of different fragrances in the mixture is not more than 10.

4. The fragrance mixture as claimed in claim 1, wherein at least one of the fragrances is present in the mixture in a concentration of at least 10% by weight, preferably at most 60% by weight.

5. The fragrance mixture as claimed in claim 1, wherein at least one of the fragrances in the mixture has a sulfur or nitrogen atom, and is preferably a nitrile group.

6. The fragrance mixture as claimed in claim 1, wherein at least one of the fragrances in the mixture has a molar mass of greater than 120 g/mol.

7. The fragrance mixture as claimed in claim 1, wherein the fragrances in the mixture have a log K_{ow} value of 1 to 10.

8. The fragrance mixture as claimed in claim 1, wherein at least 80% by weight of the fragrance molecules have dimensions of X>7 angströms, Y >4 angströms and Z>2 angströms.

9. The fragrance mixture as claimed in claim 1, wherein the fragrance mixture contains only very little solvent, if any.

10. A capsule comprising a fragrance mixture as claimed in claim 1.

11. The capsule as claimed in claim 10, wherein the capsule has an average diameter between 1 μm and 8 mm.

12. A consumer product comprising a fragrance mixture as claimed in claim 1.

13. The consumer product as claimed in claim 12, selected from the group consisting of washing and cleaning compositions, cosmetic products, perfume articles and cosmetic cleansers.

14. A consumer product comprising a capsule as claimed in claim 10.

15. The consumer product as claimed in claim 14, selected from the group consisting of washing and cleaning compositions, cosmetic products, perfume articles and cosmetic cleansers.
